(19) United States
(12) Patent Application Publication
(10) Pub. No.: US 2004/0098069 A1
(43) Pub. Date: May 20, 2004

(54) Light delivery system for improving the appearance of skin

(76) Inventors: Robert Marc Clement, Pontardawe (GB); Michael Noel Kiernan, Swansea (GB)

Correspondence Address: Gordon & Jacobson, 65 Woods End Road, Stamford, CT, 06905, US

(21) Appl. No.: 10/466,698

(22) PCT Filed: Dec. 18, 2001
(86) PCT No.: PCT/GB01/05592

(30) Foreign Application Priority Data
Dec. 22, 2000 (GB) 0031489.8

(51) Int. Cl. A61N 1/00

(52) U.S. Cl. 607/88

(57) ABSTRACT

Light from solid state devices is applied to an area of skin under conditions that produce an improvement in the appearance of an area of skin, without significantly damaging the skin. Preferred light sources are light emitting diodes (LEDs) and diode lasers, preferably selected to produce at least a substantial portion of their output in with a narrow band of wavelengths between 570 and 600 nm, more preferably within a wavelength band no greater than 15 nm, and most preferably including a substantial portion of their output at about 585 nm. A controller drives the light sources in at least one of a banked, overpulsed, and super pulsed modes. Pulsing is preferably accomplished using a pulse width of between about 1 μsec and 1 msec. A skin or other applicator preferably cooperates with the controller to provide light energy to the area of skin under conditions that produce a skin reaction without producing any significant ablation, or any other substantially deleterious effect upon the skin, especially the basal layer.

The present invention relates to a light delivery system for improving the appearance of skin.

It is well known to utilize many forms of light to improve the appearance of skin. Among lighter-skinned peoples, for example, ultra-violet light has been used to produce a tan, and to otherwise improve general skin color and tone. In many cultures light has been used to reduce acne and other skin blemishes. Light has also been used medically to stimulate wound healing, and to treat inflammatory conditions, skin ulcers, and wrinkles.

Many artificial sources of light have been used for these purposes, including both white light sources such as incandescent lamps, and narrow-band sources such as lasers and diodes. Light from dye lasers at about 585 nm, for example, has been successfully used to reduce wrinkles. See, for example U.S. Pat. No. 6,077,294. In other systems, light from diodes (LEDs and laser diodes) has been used to alleviate various musculoskeletal disorders, skin ulcers, and decrease postoperative wound healing time. See, for example, U.S. Pat. No. 5,259,380.

The various white light sources can be problematic because they emit substantial light energy outside a desired range. The undesired light can filtered out, but such filtering means that a substantial proportion of the light energy is wasted, resulting in unnecessary costs.

Some of the narrow band sources, including carbon-dioxide and YAG lasers, can produce a prodigious amount of energy, and do so rather efficiently. But such lasers can also be dangerous, and relatively expensive. Diodes and other solid state light emitters solve both of those problems, but tend to emit light at relatively low power. One solution has been to affix the diodes or light pipe directly to an area of skin, so that the emitted energy can accumulate over several hours or days (see, for example U.S. Pat. No. 5,358,503). Unfortunately such solutions have compliance and other difficulties.

Thus, there is a continuing need for systems and methods that utilize solid state light emitters to improve the appearance of skin.

The present invention provides devices and methods in which light from solid state devices is applied to an area of skin under conditions that produce an improvement in the appearance of an area of skin, without significantly damaging the skin. Although the application of light may also have an effect of treating a medical condition, the focus of this application is on improvement in the appearance of the skin.

According to a first aspect, the present invention provides light delivery system for illuminating target zones (typically skin zones) of a subject, the system comprising:

a) a plurality of light emitters capable of producing visible light;

b) an applicator in receipt of light directed from the emitters and movable to direct the light to the target zones; and,

c) a controller controlling the light emitters to operate in pulsed mode according to a predetermined regime, to deliver the light at an intensity that produces a desired effect at the subject without producing significant ablation.

In the context of the invention, a significant ablation of the skin may comprise a substantially deleterious effect upon a basal layer of skin.

According to a further aspect, the invention provides non-surgical method of obtaining a desired cosmetic skin effect, the method comprising illuminating target skin zones of a subject from a movable applicator, the light delivered from the applicator originating at a plurality of light emitters capable of producing visible light, a controller controlling the light emitters to operate in pulsed mode according to a predetermined regime, to deliver the light at an intensity that produces a desired effect at the subject without producing significant ablation.

According to a related aspect, the invention provides light system (particularly for use in a non-surgical method of obtaining a desired cosmetic skin effect), the system comprising:

a) a plurality of light emitters;

b) a controller controlling the light emitters in at least one of a banked mode, an overpulsed mode, and a superpulsed mode;

wherein;

i) the system further includes an applicator in receipt of light directed from the emitters, light from separate emitters being directed to a common light output port of the applicator, the applicator being movable to direct light to the area of skin; and/or,

ii) the emitters comprise solid state devices; and/or,

iii) the system further includes a light guide at least 1 cm long that carries the light to the area of skin.

Preferred light emitters are light emitting diodes (LEDs) and diode lasers. The light emitters are preferably selected to produce at least a substantial portion of their output in with a narrow band of wavelengths between 570 and 600 nm, more preferably within a wavelength band no greater than 15 nm, and most preferably including a substantial portion of their output at about 585 nm.

A controller may advantageously be used to operate the light emitters in at least one of a banked, overpulsed, and super pulsed modes. Two, or even all three of these modes can be used together. In addition, the controller may also operate one set of the plurality of light emitters in an overpulsed mode, a second set in a banked mode, and a third set in a super pulsed mode. Pulsing is preferably accomplished using a pulse width of between about 1 μsec and 1 msec.

A skin or other applicator preferably cooperates with the controller to provide light energy to the area of skin under conditions that produce a skin reaction without producing any significant ablation, or any other substantially deleterious effect upon the skin. It is deemed to be particularly important to achieve a desired effect without significantly damaging the basal layer of skin.

Further preferred features of the invention are presented in the appended claims and/or will be readily apparent from the description of the embodiments which follow.

the invention will now be further described in exemplary embodiments, by way of example only, with reference to the accompanying drawings( in which like numerals represent like components) in which:

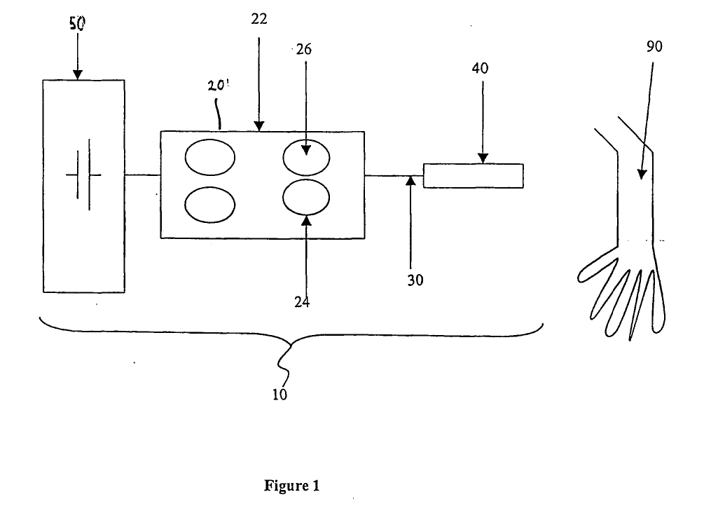

FIG. 1 is a schematic view of a light system.

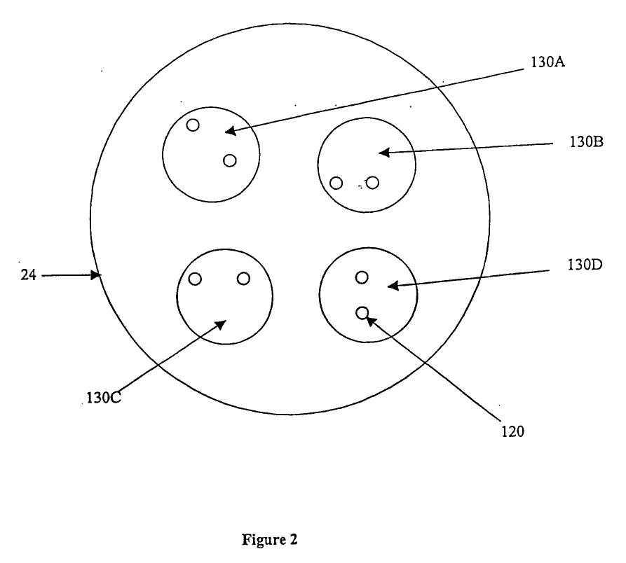

FIG. 2 is a schematic view of an array of solid state emitters.

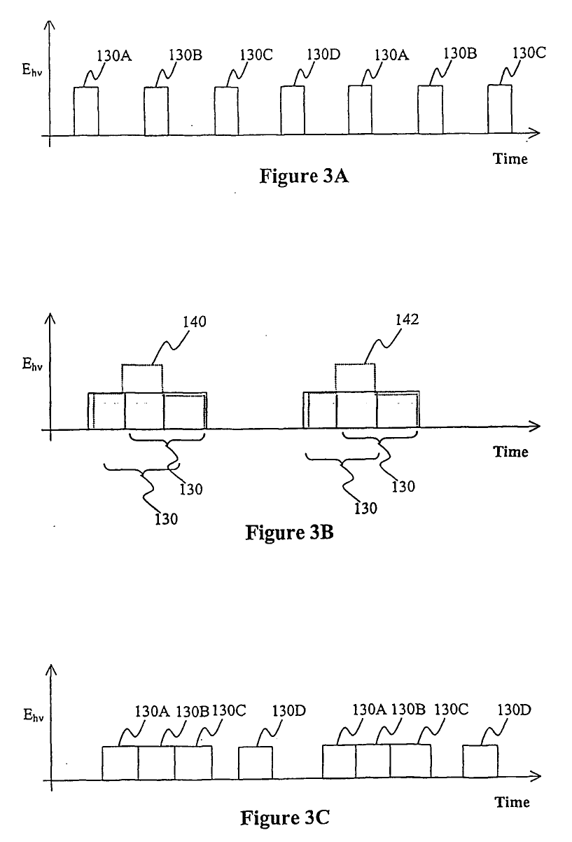

FIG. 3A is a graph of an embodiment of emitter banking.

FIG. 3B is a graph of an alternative embodiment of banking, which also depicts superpulsing.

FIG. 3C is a graph of another alternative embodiment of banking.

FIG. 3D is a graph of yet another alternative embodiment of banking, which also depicts overpulsing.

FIG. 3E is a graph of yet another alternative embodiment of banking, which also depicts superpulsing.

FIG. 3F is a graph of yet another alternative embodiment of banking, which also depicts double superpulsing.

Referring to the drawings, FIG. 1 shows a stimulator light system 10 generally including a light source housing 20, a power supply 22, a light emitter 24, an optional filter 26, an optional optic carrier 30, an applicator 40, and controller 50, all of which cooperate to apply pulses of light to the skin 90 on the arm of a subject. As used herein the term “light” is not limited to visible light. Also contemplated are other wavelengths of light including especially near UV and near IR. The term “light” is thus synonymous with the term “electromagnetic radiation”.

The housing 20 can be of any suitable size, shape, color, materials, and so forth. In FIG. 1 the light system is a floor type model. As components become miniaturized over time, however, it is contemplated that housings could be made much smaller, perhaps hand-held, and similar in overall dimensions to an electric toothbrush. Even smaller embodiments are also contemplated, including versions in which a portion of the stimulator 10 is implantable, and the remainder is carried on the outside of the body.

The power supply 22 can be of known type, conventional or otherwise, sufficient to operate the light source. It is certainly foreseen that a stimulator could be battery operated rather than relying directly on utility current.

The light emitter 24 is preferably an array of LEDs, laser diodes, or other solid state emitters having sufficient energy output in at least some suitable wavelengths. For skin applications sufficient energy means that the target tissue, whether skin or other tissue, is beneficially irradiated with 0.5 J/cm²-5.0 J/cm². Below 0.5 J/cm² it is thought that any heating or other effects would be dissipated too rapidly to have a significant effect. Above 5.0 J/cm² there is a very significant possibility of damaging the epidermis, and even the basal layer of the skin. Energy densities for other tissues would depend on the tissue.

For improving the appearance of skin, it is sometimes desirable to provide light to the target area of skin in which a substantial portion of the output falls within in a band of wavelengths no greater than 15 nm wide. One preferred band of wavelengths is between 570 nm and 600 nm, with a peak at about 585 nm. Another preferred band of wavelengths falls in a known tanning region of near UV. Unless otherwise specified, ranges throughout the specification and claims should be interpreted as inclusive of the listed endpoints. Also, as used herein the term “substantial” means at least 20%. Thus, emission of a visible light from an emitter having a substantial emission between 570 and 600 nm means that at least 20% of the total energy output of that emitter at a given point in time is between 570 and 600 nm. Where the term “substantial” is used, however, it should also be appreciated that other contemplated, and generally more preferred embodiments, would use higher percentages, including at least 40%, at least 60%, and at least 90%.

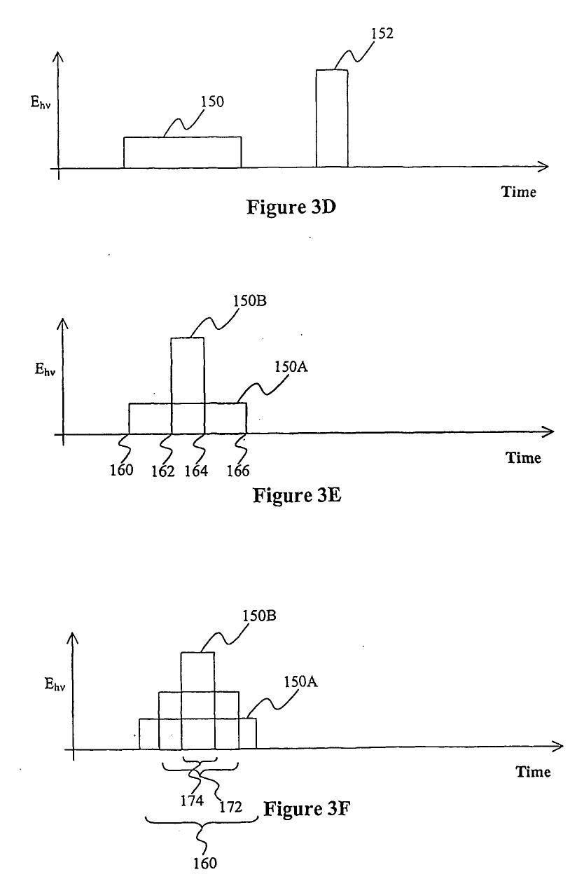

A plurality (preferably a multiplicity) of diodes are employed in light emitter 24 because individual the light emitter 24 may include hundreds or even thousands of individual diodes. It is also contemplated that the output of such diodes can be pulsed to achieve a more pronounced effect than would be the case if all such diodes were operating continuously and simultaneously. Contemplated pulsing schemes include especially those discussed below with respect to FIGS. 2, 3, and 4.

The term “light source” is used herein to include both the light emitter 24 by itself, as well as any combination of emitter and filter, or emitter and other device that collectively operate to provide a desired spectral pattern of wavelengths. Thus, it is entirely possible that a collagen stimulator (not shown) could provide a white light emitter at one end of an optic carrier, and a filter at the other end. In such an embodiment the emitter, optic carrier, and filter would collectively be considered the “light source”.

Optic carrier 30 is preferably fiber optics, but can be anything (such as an alternative waveguide) other than ordinary atmosphere that conducts the light being administered more than 10 mm. The distance limitation is intended to eliminate refraction matching or other films, as well as glass or other skin juxtaposing plates that may be included in the applicator 40. While not shown, it is also contemplated that appropriate support components would be utilized in conjunction with an optic carrier. Thus, for example, light from an LED, laser diode or other laser, white light emitter or some other emitter is likely focused into the optic carrier using one or more lenses.

The applicator 40 is used primarily to impart energy to a target tissue while imparting relatively little energy to surrounding or adjacent tissues. For larger energy densities or longer application periods this practice helps reduce collateral damage to healthy tissue, and even for lower energy densities or shorter application periods this practice may reduce pain, tingling, or other undesirable side effects. Such selective effects are contemplated to be produced largely by directing the light to a target tissue. This can be done by holding the application 40 above or on the target area of skin, and depending upon the dimensions of the target area, moving the applicator 40 accordingly. In a preferred embodiment, for example, an applicator having an elongated dispensing area (perhaps 3 mm by 10 mm), is moved back and forth along a wrinkle—thus providing relatively high exposure to the wrinkle, and relatively low exposure to the collateral tissue.

Controller 50 serves several functions, including especially operating a plurality of individual emitters according to various pulsing schemes that achieve desired light characteristics. FIG. 2 shows an array of individual solid state emitters 120 collected together to form emitter 24. In this instance the emitters 120 are grouped in four banks 130A, 130B, 130C, and 130D. In one contemplated embodiment, the controller 50 can operate the four banks sequentially, producing a pattern of emission from emitter 24 such as that depicted by line 130 in FIG. 3A. Other patters are also contemplated, however, including the pattern of FIG. 3B, in which pulses for two of the banks overlap. FIG. 3C provides yet another contemplated pattern. All of these are examples of emitters 120 operating in a banked mode.

The emitters 120 may be distinct from one another in many different ways. Emitters 120 may, for example, be distinct from one another by virtue of their being individually addressable, or addressable in groups. Emitters 120 may also or alternatively be distinct from one another in a physical sense, as being separated by an electrical non-conductor or other dielectric. Very likely, but not necessarily, all of the emitters 120 in a given emitter 24 would be produced on the same wafer to improve manufacturing efficiencies.

Banking can theoretically be used with any number of emitters 120 greater than 2, but the term is used herein in a more restricted sense to mean preferably at least 2 groupings of at least 5 emitters 120. More preferred embodiments have at least 2 groups of greater numbers of emitters 120, including at least 10 emitters, at least 25 emitters, at least 50 emitters, and even more preferably at least 100 emitters.

Regardless of whether or not emitter banking is being used, one or more of the emitters 120 can advantageously be overpulsed. As used herein, the term “overpulsing” refers to driving an emitter 120 at least 20% above its rated continuous output level, but only for short periods. Thus, if a diode were rated at x lumens for continuous output, overpulsing may use a higher than normal voltage to drive the diode at somewhere to produce light at between 2× and 5× lumens—but only for 1 msec or other short period of time. Once the diode has sufficiently cooled, it can be driven to produce yet another overpulse. Those of ordinary skill in the art will immediately recognize that the amount of overpulsing that maybe accomplished, together with the amount of rest time required between pulses, largely determines whether overpulsing is advantageous. For example, if overpulsing could only be accomplished at 2× continuous output, then there would be no marginal increase, and possibly a marginal decrease, in the output of the system if the rest time between overpulses were less than the “on” time during overpulses. But if overpulsing could be accomplished at 4× or 5× continuous output, and the rest time were equal to the “on” time, then the total output of the system would be 2.0 to 2.5 times the corresponding continuous output. With respect to the duration (i.e., pulse width) of suitable pulses, many durations are considered appropriate, and a particular duration will generally depend on a particular application. However, particularly preferred pulse widths include a pulse width of about 1 μsec to 1 msec.

FIG. 3D includes two pulses 150, 152 for a bank of emitters. The first pulse 150 is a relatively low power pulse, sustainable for a relatively long period of time. The second pulse 152 is an overpulse, operating for a relatively short period of time, and not sustainable over a long period of time. Although the cutoff between normal output and overpulsing is defined herein to be 20% above rated continuous output, it is definitely preferred that overpulsing is employed to provide at least 40% above rated continuous output, and more preferably at least 60%, at least 80%, at least 100%, at least 150%, at least 200%, at least 300%, at least 400% and at least 500%.

The term “superpulsing” is used herein to mean that at least two pulses from different emitters partially overlap so that they produce a period of high output relative to a base output. In FIG. 3B, for example, the overlapping of pulses from banks 130A and 130B add together to produce a total output characterized by the dotted line 140, and the overlapping of pulses from banks 130C and 130D add together to produce a total output characterized by the dotted line 142. In the alternative embodiment depicted in FIG. 3E, bank 130A of emitters 120 produce a relatively long pulse from time 160 to time 166. During that period, bank 130B of emitters 120 produces a relatively short overpulsed pulse from time 162 to 164. The total light output (not shown) would be the sum of both pulses.

There can be many other embodiments as well. For example, a single base pulse may be associated with two or more overlapping pulses (not shown). There may even be a double overpulsing as shown in FIG. 3F in which a first bank of emitters provides light over a relatively long period of time 170, a second bank provides light over a shorter period of time 172, and a third bank provides light over an even shorter period of time. Here, the total light output (not shown) would be the sum of all three pulses. A fourth bank (not shown), could even take over for the first bank partway through the longer period of time. Those skilled in the art should be able to readily identify many, many other permutations utilizing these concepts.

It should also be appreciated that although all of the pulses in FIGS. 3A, 3B, 3C, 3D, 3E, and 3F are shown as having virtually instantaneous ramp up and ramp down slopes, the actual ramp up and ramp down slopes in practice would not be instantaneous. Still further, it should be appreciated that the output of the emitter 24 will vary over time, and individual banks and emitters may be operated so that the total output is pulsed with complete rest periods in which virtually no light is emitted from the emitter 24, pulsed with relative rest periods having only a relatively low level of light being emitted from the emitter 24, pulsing having all sorts of different stepped, ramped, or other shapes, and so on. Nevertheless, where it is reasonable to define distinctive pulses emitted from the emitter 24, it is contemplated that such pulses would provide no more total energy Eₕᵥ to the target area of skin of between about 0.5 J/cm² and 5.0 J/cm².

Focusing again on FIG. 1, the target of light application is the skin 90 on the arm of a subject 92. Although in this instance the target is on the arm, all other skin targets are also contemplated. For example, with small area applicators one may target individual hair follicles on the face, scalp, chest, legs, pubic area, or elsewhere. In still other contemplated embodiments, the target may be the intima of a blood vessel, although in many instances it may actually be undesirable to stimulate collagen production inside blood vessels. Still further, the term “subject” is used herein to mean any higher organism including all vertebrates and especially humans.

The presently disclosed apparatus and methods have numerous applications that improve the appearance of skin, that is having a desired skin effect. Where ultra-violet light is used, for example, the improvement may involve tanning. Where light is applied at 585 nm, or other wavelength that is well absorbed by the dermis, but only poorly absorbed by the epidermis or basal layer, the improvement in skin may be in making the skin smoother. Smoother skin can be achieved, for example, through a reduction in the depth or width of a wrinkle, or through increasing collagen production under an indentation caused by acne. Another improvement may be reduction in the growth or presence of hair, such as where the application conditions are satisfactory to kill or at least significantly damage cells structurally and/or physiologically associated with growth of hair, and particularly cortical cells including hair follicle cells, papilla cells, outer and inner root sheath cells (e.g.: Huxley's layer and Henley's layer). Still another improvement in skin can be achieved indirectly, by affecting a medical condition.

With respect to methods, an improvement in an appearance of an area of skin may advantageously comprise: providing a plurality of solid state light sources that emit a visible light having a substantial emission between 570 and 600 nm; providing a controller that operates the plurality of light sources in at least one of a banked mode, an overpulsed mode, and a superpulsed mode; and providing a light guide at least 1 cm long that carries the light to the area of skin. The method may further comprise irradiating the area of skin with the light at an intensity and a duration that produces the improvement without substantially damaging a basal layer in the area of skin. It is especially contemplated that the controller and the light guide may cooperate to irradiate the area of skin under conditions that raises a temperature of a dermal layer in the area of skin to at least 70 degrees Centigrade for at least 1 ms.

As used herein the term “without significantly damaging the skin” means that no more than 5% of the cells in the area of skin being irradiated die within 5 hours of the application of the light. It is even more preferable that no more than 3%, or even 1% of such cells die within the 5 hour window after such application.

Thus, specific embodiments and applications of light therapy methods and apparatus have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context.

1. A light delivery system for illuminating target zones of a subject, the system comprising:
a) a plurality of light emitters capable of producing visible light;
b) an applicator in receipt of light directed from the emitters and movable to direct the light to the target zones; and,
c) a controller controlling the light emitters to operate in pulsed mode according to a predetermined regime, to deliver the light at an intensity that produces a desired effect at the subject without producing significant ablation.

2. A system according to claim 1, wherein the plurality of light emitters comprises a multiplicity of light emitters.

3. A system according to claim 1 or claim 2, wherein the light emitters comprise solid state light emitting devices.

4. A system according to any preceding claim, wherein the light emitters comprise diodes.

5. A system according to any preceding claim wherein the diodes emit the light in a narrow band of wavelengths between 570 nm and 600 nm.

6. A system according to any preceding claim wherein the controller is operable to operate the light emitters according to a sequenced regime.

7. A system according to claim 6, wherein the sequenced regime provides diodes to be pulsed simultaneously or in time overlap fashion giving a super-pulsed operation mode.

8. A system according to any preceding claim, wherein the emitters are operable in groups, the controller dictating operation of the relevant groups giving a banked mode of operation.

9. A system according to any preceding claim, wherein the controller is operable to operate the emitters in overpulsed mode.

10. A system according to any preceding claim, wherein the light pulses have a pulse duration substantially in the range 1 μsec to 1 msec.

11. A system according to any preceding claim, wherein the light pulses have a total pulse energy substantially in the range 0.5 J/cm²-5.0 J/cm².

12. A system according to any preceding claim, wherein the applicator includes a common light outlet for light from different emitters.

13. A non-surgical method of obtaining a desired cosmetic skin effect, the method comprising illuminating target skin zones of a subject from a movable applicator, the light delivered from the applicator originating at a plurality of light emitters capable of producing visible light, a controller controlling the light emitters to operate in pulsed mode according to a predetermined regime, to deliver the light at an intensity that produces a desired effect at the subject without producing significant ablation.

14. A method according to claim 13, wherein the desired skin effect comprises a substantially deleterious effect on a cell structurally associated with growth of a hair.

15. A method according to claim 14, wherein the cell structurally associated with growth of the hair comprises a hair follicle cell, a papilla cell, an outer root sheath cell, an inner root sheath cell, a cell of a Huxley's layer or a cell of a Henley's layer.

16. A method of setting up apparatus for use in obtaining a cosmetic non-surgical skin effect on a patient, the apparatus comprising:
a) a plurality of light emitters capable of producing visible light;
b) an applicator in receipt of light directed from the emitters and movable to direct the light to the target zones; and,
c) a controller controlling the light emitters to operate;
wherein the controller is set to operate the emitters in pulsed mode according to a predetermined regime, to deliver the light at an intensity that produces a desired effect at the subject without producing significant ablation.

17. A method according to claim 16 operating in accordance with the system of any of claims 1 to 12.

18. A light system (particularly for use in a non-surgical method of obtaining a desired cosmetic skin effect), the system comprising:
a) a plurality of light emitters;
b) a controller controlling the light sources in at least one of a banked mode, an overpulsed mode, and a superpulsed mode;
wherein;
i) the system further includes an applicator in receipt of light directed from the emitters, light from separate emitters being directed to a common light output port of the applicator, the applicator being movable to direct light to the area of skin; and/or,
ii) the emitters comprise solid state devices; and/or,
iii) the system further includes a light guide at least 1 cm long that carries the light to the area of skin.

19. A system according to claim 18 wherein the light emitters comprise diodes.

20. A system according to claim 18 or claim 19, wherein the light emitters comprise solid state light lasers.

21. A system according to claim 18 or claim 19, wherein the light emitters comprise white light emitters.

22. A system according to any of claims 18 to 21, wherein the controller operates at least a first set of the plurality of light emitters in both an overpulsed mode and a superpulsed mode.

23. A system according to any of claims 18 to 22, wherein the controller pulses at least some of the plurality of light sources to produce a pulse width of between about 1 μsec and 1 msec, and a total pulse energy of between about 0.5 J/cm²-5.0 J/cm².

24. A system according to any of claims 18 to 23, further comprising a skin applicator in collection of light directed from the emitters.

25. A system according to any of claims 18 to 24, wherein the controller operates the emitters in accordance with a predetermined regime to deliver the visible light at an intensity that produces a desired skin effect without producing a significant ablation of the skin.

26. A system according to any of claims 18 to 25, wherein the light is emitted in a narrow band of wavelengths no greater than 15 nm (or at a discrete wavelength), and that includes a wavelength between 570 and 600 nm.

27. A non-surgical method of producing an improvement in cosmetic appearance of an area of skin, comprising:
a) providing a plurality of solid state light emitters that emit a visible light having a substantial emission between 570 and 600 nm;
b) providing a controller that operates the plurality of light emitters in at least one of a banked mode, an overpulsed mode, and a superpulsed mode;
c) directing light from separate emitters to a common light output port of an applicator, the applicator being movable to direct light to the area of skin.

28. A method according to claim 27 further comprising irradiating the area of skin with the light at an intensity and a duration that produces the improvement without substantially damaging a basal layer in the area of skin.

29. A method according to claim 27 or 28 further comprising the controller and the light emitters cooperating to irradiate the area of skin under conditions that raises a temperature of a dermal layer in the area of skin to at least 70 degrees Centigrade for at least 1 ms.
